(19) United States
(12) Patent Application Publication
(10) Pub. No.: US 2008/0255416 A1
(43) Pub. Date: Oct. 16, 2008

(54) Endoscope with Miniature Imaging Arrangement

(75) Inventor: Pinhas Gilboa, Haifa (IL)

Correspondence Address: Dr. Mark Friedman Ltd.;C/o Bill Polkinghorn, 9003 Florin Way, Upper Malboro, MD, 20772, US

(73) Assignee: Super Dimension, Ltd.

(21) Appl. No.: 10/571,695

(22) PCT Filed: Jan. 26, 2006
(86) PCT No.: PCT/IL06/00113

§ 371 Date: Mar. 13, 2006

Related U.S. Application Data
(60) Provisional application No. 60/647,036, filed on Jan. 27, 2005.

(51) Int. Cl. A61B 1/05; A61B 1/06

(52) U.S. Cl. 600/110; 600/109; 600/178

(57) ABSTRACT

A miniature endoscope with an imaging arrangement associated with its distal tip portion includes an image sensor chip with a two-dimensional array of light-sensitive pixels and a lens arrangement deployed for focusing light from a field of view onto the image sensor chip so as to generate an image of a scene viewed from the distal tip portion. The lens arrangement is preferably directly affixed to the image sensor chip by a quantity of transparent adhesive. Use of bidirectional communication along data lines to the image sensor chip allows use of only four wires connecting to the chip. These and other features allow miniaturization of the endoscope to a diameter of about 2 millimeters while still generating color images of high dynamic range.

### FIELD AND BACKGROUND OF THE INVENTION

The present invention relates to endoscopes and, in particular, it concerns a miniature imaging sensor for use with particularly small diameter endoscopes.

It is known to employ endoscopes with imaging sensors to obtain images of body cavities including, but not limited to, the lungs, the stomach, the colon, and the abdomen. Endoscopes for imaging cavities within the lungs are typically referred to as “bronchoscopes”, and those for imaging within the colon are typically referred to as “colonoscopes”. All such devices with imaging capabilities for examining the inside of body cavities are referred to herein generically as “endoscopes”. Until recently, flexible endoscopes employed optical fibers to deliver the image from the distal endoscope tip to its proximal end. In recent years, video endoscopes were built, where a video camera is placed at the distal tip and the image is delivered to its proximal end via electrical wires. This arrangement improves the picture quality and makes the endoscope more flexible, since the electric wires are more flexible than the fiberscopes.

Usually the video camera has an automated gain control (AGC) that controls the exposure duration in order to avoid saturation. The AGC can be implemented internally, occupying some physical area, or alternatively the AG can be controlled from the outside via command lines. For extremely miniature sensors, i.e., with diameters below 3 millimeters, the latter is the only possible solution. The control signals need to be fed into the image sensor via dedicated lines, in addition to other lines that are required for power and video out. Therefore, the minimum number of lines required is: at least two lines for power, two lines for video output and at least one control line, giving a total of no less than 5 lines. Where active illumination is performed by light emitting elements associated with the endoscope tip, this requires an additional two lines. If three-color illumination is used, an additional four lines are required.

An endoscope includes its own light source to illuminate a scene viewed from its tip. The light typically radiates in spherical waves in which the flux density (the power per unit area) drops as the area of the sphere increases. When this is the only source of illumination, the intensity of the light illuminates the objects as a function of the inverse of the square of the distance between the source and the objects. Imaging small intrabody cavities such as small bronchial tubes requires a large dynamic sensing range because of the big difference in distances between the adjacent tissue and the relatively far distance seen at the center of this tube. Practically, since the dynamic range of the sensor is finite, in a wide viewing angle, where very close and very far tissues are seen in the same exposure, it is impossible to get an image that is free of saturation and at the same time clearly shows the dark elements of the scene. A short exposure is preferred for acquiring the image of the adjacent tissue, while more distant tissue requires a long exposure.

The incorporation of light sources into the distal tip of a very miniature endoscope often presents problems of uneven light distribution. In particular, where different colors of illuminating light are supplied from different light emitting diodes (LEDs), or via separate optic fibers from an external source, the differing geometrical positions of the light sources for the different colors often causes color imbalance between different parts of the image. A further problem in very miniature systems is the proximity of the light source to the image detector array which may lead to light leakage between the lens arrangement and the image sensor array.

There is therefore a need for a miniature endoscope which would achieve effective dispersion of illumination, reduce the number of wire connections required to the image sensor chip, and thereby facilitate implementation of an endoscope of diameter no greater than about 2 millimeters.

### SUMMARY OF THE INVENTION

The present invention is an endoscope with a miniature imaging arrangement.

According to the teachings of the present invention there is provided, an endoscope comprising: (a) an elongated flexible body having a distal tip portion; and b) an imaging arrangement associated with the distal tip portion, the imaging arrangement including: (i) an image sensor chip including a two-dimensional array of light-sensitive pixels; and (ii) a lens arrangement deployed for focusing light from a field of view onto the image sensor chip so as to generate an image of a scene viewed from the distal tip portion, wherein the lens arrangement is directly affixed to the image sensor chip by a quantity of transparent adhesive.

According to a further feature of the present invention, the lens arrangement includes a cylindrical graded-index lens. Alternatively, the lens arrangement includes a compound lens assembly.

According to a further feature of the present invention, the lens arrangement has a field of view of at least about 60°, and more preferably at least about 90°.

According to a further feature of the present invention, an area of the two-dimensional array of light-sensitive pixels is no more than half a square millimeter.

According to a further feature of the present invention, the imaging arrangement has a diameter of no more than 2 millimeters.

According to a further feature of the present invention, there is also provided: (a) at least one light source for illuminating the scene viewed from the distal tip portion; and (b) an optically dispersive medium distally overlying the light source such that the optically dispersive medium is effective to disperse illumination from the light source, thereby illuminating the scene viewed from the distal tip portion, without obscuring light reflected from the scene from reaching the lens arrangement.

According to a further feature of the present invention, the lens arrangement extends distally beyond the at least one light source, and wherein the optically dispersive medium surrounds the lens arrangement without overlying the lens arrangement.

According to a further feature of the present invention, the imaging arrangement further includes a substantially opaque medium deployed at least between the light source and the two-dimensional array of light-sensitive pixels without obscuring propagation of illumination from the light source towards the scene.

According to a further feature of the present invention, the imaging arrangement further includes a substantially transparent medium overlying both the optically dispersive medium and the lens arrangement.

According to a further feature of the present invention, the at least one light source is implemented as a plurality of light sources of different colors.

According to a further feature of the present invention, the image sensor chip is rectangular, and wherein the plurality of light sources are deployed along no more than two edges of the rectangular chip, the two-dimensional array of light-sensitive pixels being located proximal to a corner of the image sensor chip furthest from the two edges of the rectangular chip.

According to a further feature of the present invention, the at least one light source and the image sensor chip are deployed on a common circuit board.

According to a further feature of the present invention, the circuit board fits within a circular cross-section of diameter 2 millimeters.

According to a further feature of the present invention, there are also provided a plurality of wires passing along the elongated flexible body for connection to the image sensor chip and the at least one light source, the wires being connected to contact regions of the circuit board on a proximal side of the circuit board.

According to a further feature of the present invention, the image sensor chip is connected to exactly four of the plurality of wires.

According to a further feature of the present invention, there is also provided a position sensor arrangement including a plurality of sensor coils, the position sensor arrangement being deployed within the elongated flexible body near a proximal side of the circuit board.

There is also provided according to the teachings feature of the present invention, an endoscope comprising: (a) an elongated flexible body having a distal tip portion; and (b) an imaging system associated with the elongated flexible body, the imaging system including: (i) an image sensor chip including a two-dimensional array of light-sensitive pixels, the image sensor chip being associated with the distal tip portion; (ii) a controller associated with a proximal part of the elongated flexible body, the controller being electrically associated with the image sensor chip via no more than two communication wires extending along the elongated flexible body, wherein the image sensor chip is configured to be responsive to a timing signal generated by the controller to perform a read cycle of the two-dimensional array of light-sensitive pixels in a rolling-shutter mode and to transmit a single frame of image data to the controller, wherein both the timing signal and the image data are transmitted via the no more than two communication wires.

According to a further feature of the present invention, the timing signal is a frame request signal, and wherein the image sensor chip is configured to wait after transmitting the single frame of image data until receiving a subsequent frame request signal from the controller.

According to a further feature of the present invention, the controller is configured to actuate the image sensor chip to generate pairs of similar frames with different exposure durations, the controller being further configured to co-process the pairs of similar frames to derive an enhanced frame having a dynamic range greater than each of the pair of similar frames.

According to a further feature of the present invention, there is also provided an illumination system deployed for illuminating a scene viewed from the distal tip portion, the illumination system being configured for selectively illuminating the scene with each of three different colors of visible light, wherein the illumination system is controlled by the controller such that the controller derives an enhanced frame from a pair of similar frames with different exposure durations sampled for each of the three different colors, the controller being further configured to combine the enhanced frames to generate a color image.

According to a further feature of the present invention, there is also provided an illumination system including at least one light emitting diode associated with the distal tip portion, the light emitting diode and the image sensor chip being mounted on a common circuit board.

According to a further feature of the present invention, there is also provided a quantity of an optically dispersive medium overlying the at least one light emitting diode so as to disperse illumination from the light emitting diode.

### BRIEF DESCRIPTION OF THE DRAWINGS

The invention is herein described, by way of example only, with reference to the accompanying drawings, wherein:

FIG. 1 is a schematic view of an endoscope, constructed and operative according to the teachings of the present invention;

FIG. 2 is an enlarged schematic isometric view of a distal tip portion of the endoscope of FIG. 1 with an outer cover removed to reveal an imaging arrangement, constructed and operative according to the teachings of the present invention;

FIG. 3 is a further enlarged schematic isometric view of the imaging arrangement of FIG. 2;

FIG. 4 is a partially exploded isometric view of the imaging arrangement of FIG. 2;

FIG. 5 is a schematic cross-sectional view taken through the imaging arrangement of FIG. 2 illustrating a layered structure of encapsulation of the imaging arrangement according to a further feature of the present invention;

FIG. 6A is a schematic isometric view of an apparatus for use in assembly of the imaging arrangement of FIG. 2, the apparatus being shown during a chip alignment step;

FIG. 6B is an enlarged view of a region of FIG. 6A designated “B”;

FIG. 6C is a schematic isometric view of the apparatus of FIG. 6A shown during a lens attachment step;

FIG. 7 is a graphic representation of a relation between pixel output signal and scene brightness for two different durations of exposure, labeled “T₁” and “T₂”;

FIG. 8 is a graphic representation of a relation between pixel output signal and scene brightness derived from a pair of exposures of two different durations of exposure as illustrated in FIG. 7;

FIG. 9 is a schematic representation of the layout of a CMOS imaging sensor chip from the imaging arrangement of FIG. 2;

FIG. 10 is a functional representation of the operation of a CMOS image sensor pixel element from the chip of FIG. 9;

FIG. 11 is a schematic representation of a first communication arrangement for bidirectional communication with the imaging sensor arrangement of FIG. 2 according to the teachings of the present invention; and

FIG. 12 is a schematic representation of a second communication arrangement for bidirectional communication with the imaging sensor arrangement of FIG. 2 according to the teachings of the present invention.

### DESCRIPTION OF THE PREFERRED EMBODIMENTS

The present invention is an endoscope with a miniature imaging arrangement.

The principles and operation of endoscopes according to the present invention may be better understood with reference to the drawings and the accompanying description.

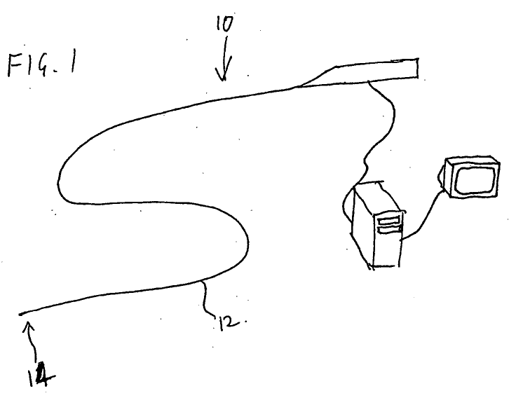

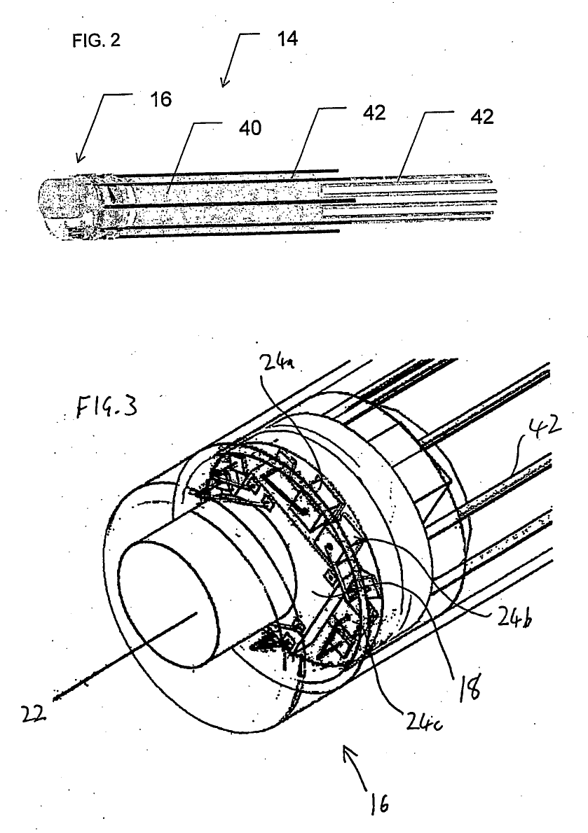

Referring now to the drawings, FIG. 1 shows a general view of an endoscope, generally designated 10, constructed and operative according to the teachings of the present invention. The endoscope has an elongated flexible body 12 with a distal tip portion 14. As seen in FIGS. 2-5, an imaging arrangement 16 is associated with distal tip portion 14. Imaging arrangement 16 includes an image sensor chip 18 including a two-dimensional array 20 of light-sensitive pixels, and a lens arrangement 22 deployed for focusing light from a field of view onto image sensor chip 18 so as to generate an image of a scene viewed from the distal tip portion.

It is a particularly preferred feature of the present invention that elongated flexible body 12 in general, and imaging arrangement 16 in particular, is a small caliber device, preferably of outer diameter no more than 3 millimeters, and most preferably of outer diameter no more than about 2 millimeters. This allows the endoscope to be introduced into small body cavities and passages which are normally inaccessible to conventional endoscopes of larger dimensions. For example, according to certain preferred embodiments, the endoscope may be inserted via a working lumen of a conventional bronchoscope and advanced into bronchial airways beyond the reach of the conventional bronchoscope where conventional procedures would require working “blind”. The miniaturization of the imaging arrangement of an endoscope to such small dimensions poses a number of significant problems of implementation. The present invention relates primarily to effective solutions for a number of such problems.

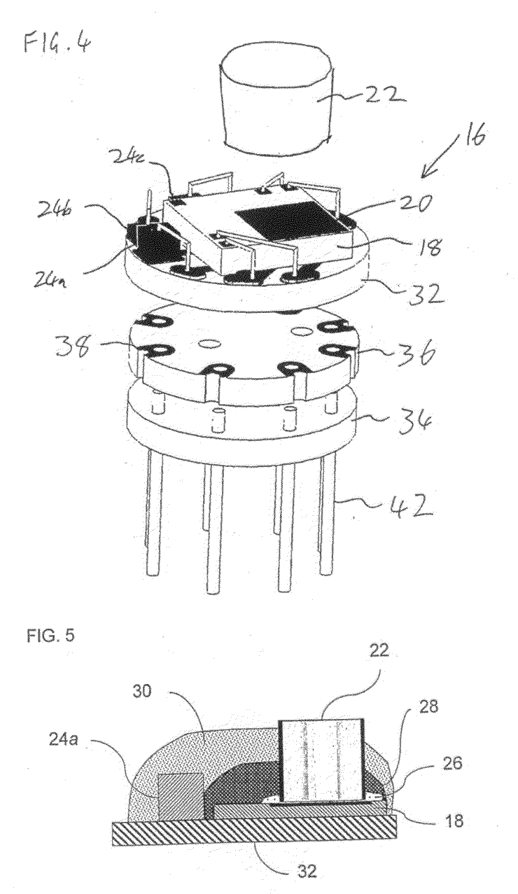

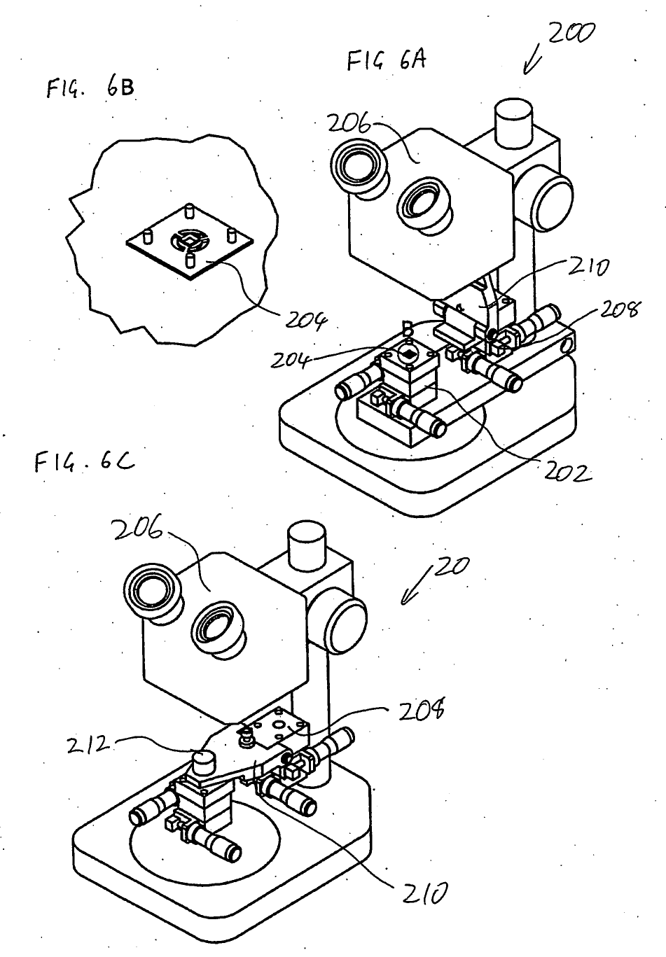

Specifically, one issue plaguing such miniature implementations of an imaging sensor is how to achieve and maintain correct alignment of lens arrangement 22 with sensor array 20 where both the lens arrangement and the sensor chip have dimensions of the order of a millimeter or less. According to one aspect of the present invention, this issue is addressed by directly affixing lens arrangement 22 to image sensor chip 18 by a quantity of transparent adhesive 26 (FIG. 5, shown with slight excess at the sides of the lens). According to a further complementary aspect of the present invention, there is provided an apparatus (FIGS. 6A-6C) for facilitating affixing of lens arrangement 22 to image sensor chip 18 in correct alignment with array 20.

A further problem plaguing miniature implementations of an imaging sensor is unwanted or uneven distribution of illuminating radiation. Endoscope 10 includes at least one source of illumination, such as light emitting diodes (“LEDs”) 24a, 24b and 24c. Due to the proximity of the light sources to the sensor array, light may leak around the base of lens arrangement 22, thereby degrading image quality. Furthermore, particularly where separate colored light sources are deployed asymmetrically relative to the lens arrangement, illumination tends to be non-uniform across the viewed scene and dissimilar between the different colors, leading to color imbalance in the output image. According to one further aspect of the present invention, the issue of light leakage is addressed as illustrated in FIG. 5 by deploying a substantially opaque medium 28 between the light sources 24a, 24b and 24c and the two-dimensional array 20 of light-sensitive pixels without obscuring propagation of illumination from the light sources 24a, 24b and 24c towards the scene to be viewed. According to a second further aspect of the present invention, the problem of non-uniform light distribution is addressed, also as illustrated in FIG. 5, by deploying an optically dispersive medium 30 distally overlying light sources 24a, 24b and 24c such that medium 30 is effective to disperse illumination from the light source, thereby illuminating the scene viewed from the distal tip portion, without obscuring light reflected from the scene from reaching lens arrangement 22.

A further issue problematic for the miniaturization of endoscope 10 is the number of connection wires which need to be attached to imaging arrangement 16. According to a still further aspect of the present invention, the number of connections to image sensor chip 18 is reduced to a total of four: two power connections and two communication connections. For this purpose, the present invention provides both a system and a method for operating an image sensor system in which two communication connections are used bi-directionally for both a frame request to the imaging arrangement and for outputting data from the imaging arrangement.

These and other aspects of the present invention will be better understood from the following detailed description.

Before addressing features of the present invention, it will be helpful to define certain terminology as used herein in the description and claims. Firstly, reference is made to “light” and “illumination”. These terms are used herein to refer generically to all parts of the electromagnetic spectrum which can be detected by low-cost silicon-based image sensors, such as CMOS sensors. This includes all of the range of wavelengths from near-ultraviolet through to near-infrared (wavelengths of between 0.25 microns and 1.1 microns). Most preferably, visible light in the range of wavelengths from about 0.4 microns to about 0.75 microns is used. The light may be monochromatic, or may contain a number of different colors simultaneously or alternately. Broad spectrum white light may also be used. Certain particularly preferred options for illumination will be discussed below.

The term “light source” is used herein to refer to any component which releases light from imaging arrangement 16 towards the scene to be viewed. The source may either generate light, as in the case of a LED, or may convey light from a remote location, as in the case of an optic fiber conveying light from a source associated with a proximal part of the endoscope body.

The scene viewed by imaging arrangement 16 may be any scene visible from the distal tip portion 14 of the endoscope. Typically, the invention is implemented as a forward-looking imaging arrangement where the optical axis of lens arrangement 22 is roughly parallel to a central axis of distal tip portion 14. It should be noted, however, that other implementations, such as a side-looking endoscope, also fall within the scope of the present invention.

Turning now to the features of imaging arrangement 16 in more detail, according to a first preferred option, lens arrangement 22 includes a cylindrical graded-index (“GRIN”) lens. Alternatively, a miniature compound lens assembly made from injection molded polymer components (typically polycarbonate) is used. In either case, lens arrangement 22 is preferably cylindrical with a total height of no more than about 1.5 millimeter and a diameter of no more than about one millimeter. The field of view of lens arrangement 22 is preferably at least about 60°, and most preferably at least about 90°.

Image sensor chip 18 is preferably a CMOS chip with major dimensions of roughly one millimeter square. Roughly half of the surface area (e.g., a square of side roughly 0.7 mm) accommodates the sensor array 20 while the remaining surface is used for the associated electronic components for reading the array, shown schematically in FIG. 9, as is known in the art. Thus, the area of two-dimensional array 20 is typically no more than about half a square millimeter (i.e., 5×1⁻⁷ m²). It has been found that these dimensions, with current production technology commercially available, are sufficient for implementing a monochrome sensor array of resolution approximately 100×100 pixels.

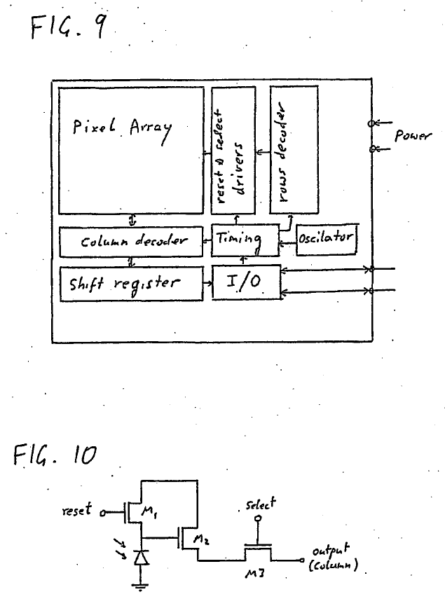

Each pixel is structured from the classical three-transistor architecture as described in FIG. 10. Switch M1 is effective to reset the pixel and to charge the photodiode. Impinging light then discharges the diode and creates a voltage difference compared to the reset level. The photodiode voltage is read out via transistor M2 that acts as a source follower. The output voltage is fed into the column readout. The sensor operates in a ‘rolling shutter’ mode, i.e., each row in turn is reset during reading of that row. The rest of the components of image sensor chip 18 illustrated in FIG. 9 are the necessary peripheral logic to read out the array. The control logic includes a vertical address decoder, horizontal shift register, column amplifiers to remove fixed pattern noise and a section generating the required frame and line pulses controlled by an on-chip oscillator. Since the area of the chip is extremely small, it is not possible to incorporate more sophisticated features such as programmable gain, offset adjustment, internal automatic gain control etc. on the chip. Instead, the more sophisticated control is preferably implemented by use of a separate controller located remote from the sensor and connected by wires passing along the endoscope. This subdivision requires bidirectional communication between the controller and the imaging arrangement, as will be discussed further below.

As discussed above, light from a point source is radiant on a surface in front of the source with a flux density that varies inversely as the square of the distance between the source and the surface. For instance, the difference between the flux density radiated on two surfaces, one located 1 mm from the source and the other 20 mm from the source, is a factor of 400. The dynamic rage of the image sensors is finite. Often, when an image combines areas of very high brightness and very low brightness, the result is either saturation of the brighter areas or poor display of the darker areas. A miniature endoscope located in a very small intra-bodily tube, for instance the. small bronchi, essentially suffers from this effect. The image combines very close and very far portions of tissue, from adjacent tissue at the side to 25-30 mm along the center of the tube. Consequently there is very big difference between the light reflected from the adjacent tissue and the far portions. An image of such tube necessarily suffers from either saturation of the very proximal tissue or too poor noisy quality of display of the dark areas at the center of that tube.

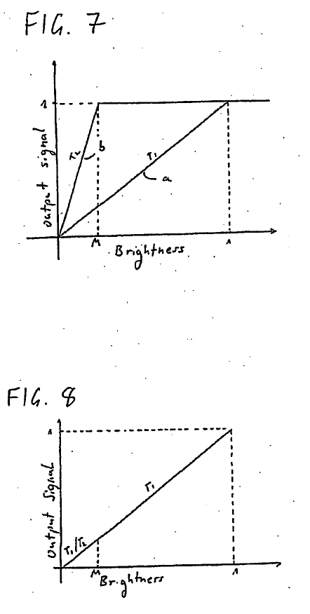

To address this problem, each video frame is preferably exposed with two different exposure durations. FIG. 7 shows the output response of two such exposures. For a scene containing a wide range of brightness levels, a sufficiently short exposure duration T₁ avoids saturation over the entire scene. A second T₂ exposure, longer than T₁ by a factor of about 10 times, produces a better image of the darker areas of the image, but gets into saturation for picture elements brighter than some value M. These two images are then combined as shown in FIG. 8 where the pixels above brightness level M are taken from exposure T₁, and the elements darker than M are taken from exposure T₂ with their values scaled by factor T₁/T₂ to correct for the exposure difference.

In order to achieve color imaging using a monochrome sensor array, the present invention preferably employs a plurality of light sources of different colors, and particularly, red, green and blue (RGB) sources, illustrated here as LEDs 24a, 24b and 24c. By capturing frames using sequential illumination by each one of the primary colors alone, the monochrome frames each represent one channel of a color RGB image.

Combining the double exposure technique with the three separate color illuminations yields six exposures for each color frame. Each exposure has its own duration, controlled externally by switching on and off the corresponding illumination source according to exposure control methods to be described below. The pairs of long/short exposures are first combined as described above, The final color image is then the combination of the double exposures for each of the three RGB exposures. First, each of the three basic RGB layers is collected according to the double exposures technique described above. Then the final color image is achieved by chromatic correction done by multiplication of each color layer in the white balance constants. Exact synchronization is needed to switch the LEDs on and off to get homogenous exposure over the entire frame each of the entire frame and to avoid mixing of colors between frames. A rolling-shutter read cycle is preferably triggered between exposures, i.e., when the scene is dark due to lack of illumination, to avoid mixing of the color frames.

Any image sensor suffers to some extent from fixed pattern noise that arises from the variation of the offset and gain of the individual pixels. Data to correct these variations can be measured and stored in a memory, for example an EPROM, implemented as part of the endoscope. Additional distortion may result from any uncorrected chromatic aberrations from lens arrangement 22. Since every color has its own layer, geometric distortions due to color shifts can be corrected mathematically using geometric transformations. The constants for these transformations can be calibrated individually and stored in the memory of the endoscope.

Turning now to the issue of light leakage and distribution, as a first precaution to minimize light leakage from the light sources directly to the sensor array, the light sources are preferably located as far away from the sensor array as allowed by the dimensions of the imaging arrangement. Thus, in the preferred example shown here (for example in FIG. 5), image sensor chip 18 is rectangular, and more particularly square. Light sources 24a, 24b and 24c are deployed along no more than two edges of rectangular chip 18, and sensor array 20 is located proximal to a corner of image sensor chip 18 furthest from the aforementioned two edges.

As a further precaution against light leakage, imaging arrangement 16 preferably further includes a quantity of a substantially opaque medium 28 deployed at least between light sources 24a, 24b and 24c and sensor array 20 in such a manner as to avoid obscuring propagation of illumination from the light source towards the scene.

In order to improve uniformity of illumination, and more particularly, to render spatial distribution of light from the different color LEDs more similar, imaging arrangement 16 preferably also includes an optically dispersive medium 30 distally overlying light sources 24a, 24b and 24c such that optically dispersive medium 30 is effective to disperse illumination from the light sources 24a, 24b and 24c, thereby illuminating the scene viewed from the distal tip portion, without obscuring light reflected from the scene from reaching lens arrangement 22. This may advantageously be achieved by ensuring that lens arrangement 22 extends distally beyond light sources 24a, 24b and 24c, and deploying optically dispersive medium 30 surrounding lens arrangement 30 without overlying the lens. Suitable optically dispersive media include, but are not limited to, adhesives described commercially as “fogged epoxy” and clear adhesives with admixtures of small crystalline or otherwise particulate solids which cause suitable scattering of light.

Optionally, imaging arrangement 16 may further include a substantially transparent medium (not shown) overlying both optically dispersive medium 30 and lens arrangement 22 to encapsulate and protect imaging arrangement 16.

Preferably, imaging arrangement 16 includes a common circuit board 32 which provides a common mounting structure for light sources 24a, 24b and 24c and image sensor chip 18. Optionally, the light sources may be raised above the surface level of the circuit board by use of a support block not shown) in order to reduce or avoid casting of an illumination shadow by the lens assembly. Circuit board 32 preferably fits within a circular cross-section of diameter 2 millimeters. Most preferably, a roughly circular circuit board of diameter no more than about 1.8 millimeters is used. This facilitates construction of an endoscope with an external diameter no greater than about 2 millimeters.

Electrical wires 42 for supplying power to light sources 24a, 24b and 24c and for power supply and data transfer to and from image sensor chip 18 pass along elongated flexible body 12. In order to facilitate connection of these wires to their respective devices without taking up valuable surface space on the top of the circuit board, connections of the wires are preferably achieved via connection to contact regions of the circuit board on a proximal side of the circuit board, i.e., facing away from the viewing direction. Connections between these contact regions and the components on the circuit board are achieved via through-bores in the circuit board, as is known in the art. Alignment of the wires for attachment to the corresponding contact pads may be achieved using various techniques. By way of one particularly preferred but non-limiting example illustrated in FIG. 4, the wires are held in the required formation by a positioning disc 34 configured to leave a small unclad length of each wire projecting. An adapter block 36 is formed with peripheral channels within which the ends of the wires are attached with a small drop of conductive adhesive or solder. The peripheral channels are formed with conductive coatings which are electrically connected to contact pads 38. Contact pads 38 are deployed so as to align with corresponding contact regions on the rear face of circuit board 32. Adapter block 36 and circuit board 32 are typically connected with a drop conductive adhesive or solder applied to each of contact pads 38.

Preferably, distal tip portion 14 includes a position sensor arrangement 40 (FIG. 2), including a plurality of sensor coils, deployed near a proximal side of the circuit board. Position sensor arrangement 40 is preferably implemented as a sensor arrangement of a six-degrees-of-freedom position measurement system, and most preferably according to the teachings of U.S. Pat. No. 6,188,355 and published PCT Application Nos. WO 00/10456 and WO 01/67035, all of which are hereby incorporated by reference. The position sensor arrangement 40 provides tracking of the position of imaging arrangement 16 within the body, thereby facilitating navigation of the endoscope and integration of the imaging data with other available sources of information.

As mentioned above, one of the factors problematic for miniaturization of an endoscope is the number of wires 42 which extend along the flexible body and must be connected to the imaging arrangement. In order to reduce the number of wires as much as possible, it is a particularly preferred feature of certain embodiments of the present invention that image sensor chip 18 is connected to exactly four wires. Operation of imaging arrangement 16 is controlled by a controller, which may be implemented as a dedicated electronics unit or as part of a general purpose computer system 44 (FIG. 1), associated with a proximal part of elongated flexible body 12. In order to achieve four-wire connection of the image sensor chip, the controller is electrically associated with image sensor chip 18 via no more than two communication wires 42 extending along the elongated flexible body 12. Communication is preferably achieved bi-directionally, by configuring image sensor chip 18 to be responsive to a frame request signal generated by the controller to perform a read cycle of the two-dimensional array of light-sensitive pixels in a rolling-shutter mode, and to transmit a single frame of image data to the controller. Both the frame request signal and the image data are transmitted via one or both of the two communication wires. Preferably, at least the image data is transmitted on both wires using a differential signal in order to minimize data corruption.

Synchronization of the read cycle of image sensor chip 18 is preferably controlled by the controller. Thus, image sensor chip 18 is preferably configured to wait after transmitting the single frame of image data until receiving a subsequent frame request signal from the controller. In practice, since the imaging arrangement operates in darkness, exposure control is preferably primarily achieved by controlling the activation time of the illumination sources, also controlled by the controller. However, for efficient use of time, it is preferable that a read cycle of the chip is initiated immediately after each exposure is completed. Thus, the controller is preferably configured to generate frame request signals at the end of pairs of unequal periods, corresponding to the aforementioned short and long exposure times for each color illumination. As explained earlier, pairs of similar frames with different exposure durations are generated, and are co-processed by the controller to derive an enhanced frame having a dynamic range greater than each of the pair of similar frames. In the aforementioned preferred color imaging implementation, the controller is preferably also configured to combine the enhanced frames for each of the three colors of illumination to generate a color image.

One non-limiting example of a simple electronic implementation for the bi-directional communication between image sensor chip 18 and the controller is illustrated in FIG. 11. The electronics of image sensor chip 18 is here designated 500 while the external electronics (part of the controller) is designated 550. The image sensor chip operates in rolling shutter mode with external synchronization via a frame request from the controller transmitted along the video output line 510, which is preferably a dual differential line.

The video out signal, either digital or analog, is derived by driver 506 and transmitted through the closed switch 504 and along line 510 to a receiver 552, which delivers the video signal 556 to its final destination (e.g., computer system 44 or any other required equipment). After a full frame is transferred, switch 504 is opened, and the image sensor waits for a frame request command. In this state, the cells collect photon electrons. For actuating a frame request, switch 554 is closed, grounding line 510, and thereby changing the output of amplifier 502 so as to activate the next read cycle of the image array. The read cycle also resets the pixels during the image output data transfer, row by row. The external electronics 550 can be controlled by a PC, micro-controller or any other suitable state machine.

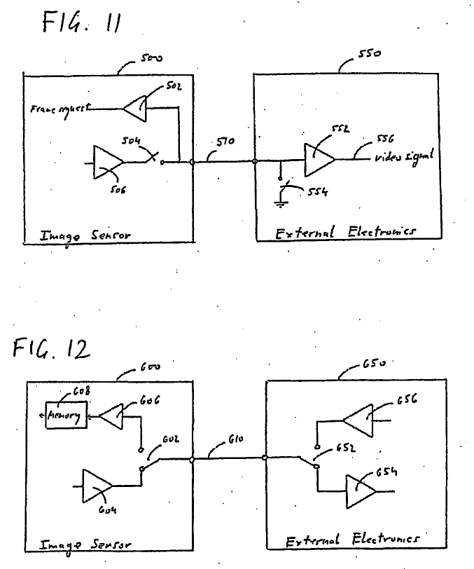

A second non-limiting example of a more sophisticated architecture is described in FIG. 12. Here, the image sensor electronics 600 and external electronics 650 are connected via a bidirectional communication line 610, again preferably a dual differential line. During the video image data transmission, a first switch 602 connects the output driver 604 to line 610 and a second switch 652 connects line 610 to an amplifier 654 to receive the signal. After completion of transfer of a frame, switches 602 and 652 change state, allowing driver 656 to send digital data to the image sensor, received by amplifier 606 and stored into memory 608. This digital data is used to control the delay between sequential video frames using count down counter. Optionally this port can serve also for other control commands.

In either of the above cases, a practical implementation of the electronic arrangement is well within the capabilities of one ordinarily skilled in the art and will not be addressed here in further detail.

Turning finally to FIGS. 6A-6C, there is shown an apparatus 200 useful for assembly of imaging arrangement 16, and in particular, for correctly aligned attachment of lens arrangement 22 by clear adhesive to the sensor array of chip 18. Apparatus 200 has a first adjustable platform 202 with a clamping surface 204 (FIG. 6B) for gripping circuit board 32 which carries image sensor chip 18. Adjustable platform 202 is mounted beneath a microscope 206 so that chip 18 can be viewed and so that the sensor array 20 can be centered under a reticule of microscope 206 by adjustment of platform 202. Apparatus 200 further includes an adjustable support 208 to which a hinged flap 210 is hingedly mounted. Hinged flap 210 includes a lens holder 212 for clamping lens arrangement 22 in a well-defined centered position. Adjustable support 208 also allows adjustment of the position of hinged flap 210 for centering a marker on the rear (upper) side of lens holder 212 relative to the microscope reticule when the flap is in its lowered position (FIG. 6C).

Before use, hinged flap 210 is lowered to the position of FIG. 6C and adjustable support 208 is adjusted until lens holder 212 is centered relative to the microscope reticule. Flap 210 is then raised to the position of FIG. 6A, and lens arrangement 22 is inserted into lens holder 212. Circuit board 32 is clamped to clamping surface 204 and adjustable platform 202 is adjusted to center the sensor array 20 relative to the microscope reticule. A small quantity of clear adhesive is then applied to the end of lens arrangement 22 and hinged flap 210 is gently lowered to bring lens arrangement 22 into contact with the sensor array where it is left until dry. Hinged flap 210 is preferably configured to apply a predefined contact pressure between the lens arrangement and the sensor array, thereby helping to ensure that the lens arrangement seats itself squarely against the chip surface.

It will be appreciated that the above descriptions are intended only to serve as examples, and that many other embodiments are possible within the scope of the present invention as defined in the appended claims.

What is claimed is:

1. An endoscope comprising:
(a) an elongated flexible body having a distal tip portion; and
(b) an imaging arrangement associated with said distal tip portion, said imaging arrangement including:
(i) an image sensor chip including a two-dimensional array of light-sensitive pixels; and
(ii) a lens arrangement deployed for focusing light from a field of view onto said image sensor chip so as to generate an image of a scene viewed from said distal tip portion,
wherein said lens arrangement is directly affixed to said image sensor chip by a quantity of transparent adhesive.

2. The endoscope of claim 1, wherein said lens arrangement includes a cylindrical graded-index lens.

3. The endoscope of claim 1, wherein said lens arrangement includes a compound lens assembly.

4. The endoscope of claim 1, wherein said lens arrangement has a field of view of at least about 60°.

5. The endoscope of claim 1, wherein said lens arrangement has a field of view of at least about 90°.

6. The endoscope of claim 1, wherein an area of said two-dimensional array of light-sensitive pixels is no more than half a square millimeter.

7. The endoscope of claim 1, wherein said imaging arrangement has a diameter of no more than 2 millimeters.

8. The endoscope of claim 1, further comprising:
(a) at least one light source for illuminating the scene viewed from said distal tip portion; and
(b) an optically dispersive medium distally overlying said light source such that said optically dispersive medium is effective to disperse illumination from said light source, thereby illuminating the scene viewed from said distal tip portion, without obscuring light reflected from the scene from reaching said lens arrangement.

9. The endoscope of claim 8, wherein said lens arrangement extends distally beyond said at least one light source, and wherein said optically dispersive medium surrounds said lens arrangement without overlying said lens arrangement.

10. The endoscope of claim 8, wherein said imaging arrangement further includes a substantially opaque medium deployed at least between said light source and said two-dimensional array of light-sensitive pixels without obscuring propagation of illumination from said light source towards the scene.

11. The endoscope of claim 8, wherein said imaging arrangement further includes a substantially transparent medium overlying both said optically dispersive medium and said lens arrangement.

12. The endoscope of claim 8, wherein said at least one light source is implemented as a plurality of light sources of different colors.

13. The endoscope of claim 12, wherein said image sensor chip is rectangular, and wherein said plurality of light sources are deployed along no more than two edges of said rectangular chip, said two-dimensional array of light-sensitive pixels being located proximal to a corner of said image sensor chip furthest from said two edges of said rectangular chip.

14. The endoscope of claim 8, wherein said at least one light source and said image sensor chip are deployed on a common circuit board.

15. The endoscope of claim 14, wherein said circuit board fits within a circular cross-section of diameter 2 millimeters.

16. The endoscope of claim 14, further comprising a plurality of wires passing along said elongated flexible body for connection to said image sensor chip and said at least one light source, said wires being connected to contact regions of said circuit board on a proximal side of said circuit board.

17. The endoscope of claim 16, wherein said image sensor chip is connected to exactly four of said plurality of wires.

18. The endoscope of claim 14, further comprising a position sensor arrangement including a plurality of sensor coils, said position sensor arrangement being deployed within said elongated flexible body near a proximal side of said circuit board.

19. An endoscope comprising:
(a) an elongated flexible body having a distal tip portion; and
(b) an imaging system associated with said elongated flexible body, said imaging system including:
(i) an image sensor chip including a two-dimensional array of light-sensitive pixels, said image sensor chip being associated with said distal tip portion;
(ii) a controller associated with a proximal part of said elongated flexible body, said controller being electrically associated with said image sensor chip via no more than two communication wires extending along said elongated flexible body,
wherein said image sensor chip is configured to be responsive to a timing signal generated by said controller to perform a read cycle of said two-dimensional array of light-sensitive pixels in a rolling-shutter mode and to transmit a single frame of image data to said controller,
wherein both said timing signal and said image data are transmitted via said no more than two communication wires.

20. The endoscope of claim 19, wherein said timing signal is a frame request signal, and wherein said image sensor chip is configured to wait after transmitting said single frame of image data until receiving a subsequent frame request signal from said controller.

21. The endoscope of claim 20, wherein said controller is configured to actuate said image sensor chip to generate pairs of similar frames with different exposure durations, said controller being further configured to co-process said pairs of similar frames to derive an enhanced frame having a dynamic range greater than each of said pair of similar frames.

22. The endoscope of claim 21, further comprising an illumination system deployed for illuminating a scene viewed from said distal tip portion, said illumination system being configured for selectively illuminating the scene with each of three different colors of visible light, wherein said illumination system is controlled by said controller such that said controller derives an enhanced frame from a pair of similar frames with different exposure durations sampled for each of said three different colors, said controller being further configured to combine said enhanced frames to generate a color image.

23. The endoscope of claim 20, further comprising an illumination system deployed for illuminating a scene viewed from said distal tip portion, said illumination system being configured for selectively illuminating the scene with each of three different colors of visible light, wherein said illumination system is controlled by said controller such that said controller samples frames for each of said three different colors, said controller being further configured to combine said frames to generate a color image.

24. The endoscope of claim 20, further comprising an illumination system including at least one light emitting diode associated with said distal tip portion, said light emitting diode and said image sensor chip being mounted on a common circuit board.

25. The endoscope of claim 24, further comprising a quantity of an optically dispersive medium overlying said at least one light emitting diode so as to disperse illumination from said light emitting diode.
